(19) United States
(12) Patent Application Publication
(10) Pub. No.: US 2016/0081616 A1
(43) Pub. Date: Mar. 24, 2016

(54) APPARATUS AND METHOD FOR PROCESSING ELECTROENCEPHALOGRAM, AND SLEEP MONITORING WEARABLE DEVICE

(71) Applicant: BOE TECHNOLOGY GROUP CO., LTD., Beijing (CN)

(72) Inventor: Hui Li, Beijing (CN)

(73) Assignee: BOE TECHNOLOGY GROUP CO., LTD., Beijing (CN)

(21) Appl. No.: 14/647,309

(22) PCT Filed: Jan. 13, 2015
(86) PCT No.: PCT/CN2015/070621

§ 371 Date: May 26, 2015

(30) Foreign Application Priority Data
Sep. 23, 2014 (CN) 201410490680.X

(51) Int. Cl. A61B 5/00; A61B 5/0205; A61B 5/04; A61B 5/0476

(52) U.S. Cl. CPC A61B 5/4812; A61B 5/0476; A61B 5/02055; A61B 5/04012; A61B 5/742; A61B 5/6803; A61B 5/024

(57) ABSTRACT

The present disclosure relates to the field of electronic technology, and provides an EEG processing apparatus and method and a sleep monitoring wearable device, which can reduce complexity of sleep staging. The EEG processing method comprises: obtaining EEG data to be processed (101); analyzing the EEG data to be processed to obtain a predetermined number of segments which do not overlap each other (102); obtaining a maximum value and a minimum value of a peak-to-peak value in each of the segments (103); normalizing a connection line between the maximum value and the minimum value of the peak-to-peak value in at least one of the segments to obtain upper end points and lower end points of the connection line (104); calculating statistic characteristic values of an upper sideband curve formed by the upper end points to obtain a box plot series (105); and obtaining a sleep staging result based on the box plot series (106).

### TECHNICAL FIELD OF THE DISCLOSURE

The present disclosure relates to the field of electronic technology, in particular, to an apparatus and method for processing electroencephalogram (EEG), and a sleep monitoring wearable device.

### BACKGROUND

Clinically, the polysomnogram (PSG) is used to perform sleep monitoring. In such a method, it needs to place multiple electrodes or sensors on body surface, all night monitoring is performed by signals collected by the electrodes or sensors to obtain a PSG, and analysis on sleep time, sleep staging and sleep efficiency is then performed based on the PSG, such as to obtain objective recognition and evaluation of the sleep quality.

Currently, the monitoring of sleep EEG is performed by arranging multiple electrodes on the scalp to collect EEC and performing the sleep EEG staging based on the R&K rule (Rechtschaffen & Kales). At present, the mainstream methods for sleep staging include wavelet transform approach, artificial neural network approach, and approximate entropy acquisition, among others. These methods are complex in computation, and need to precisely extract sleep EEG information. Therefore, it is difficult to realize sleep staging.

### SUMMARY

Embodiments of the present disclosure provide an EEG processing apparatus and method and a sleep monitoring wearable device, which can reduce complexity of sleep staging.

Accordingly, embodiments of the present disclosure employ the following technical solutions.

In one aspect, there is provided an EEG processing method comprising:

obtaining EEG data to be processed;

analyzing the EEG data to be processed to obtain a predetermined number of segments which do not overlap each other;

obtaining a maximum value and a minimum value of a peak-to-peak value in each of the segments;

normalizing a connection line between the maximum value and the minimum value of the peak-to-peak value in at least one of the segments to obtain upper end points and lower end points of the connection line;

calculating statistic characteristic values of an upper sideband curve formed by the upper end points to obtain a box plot series; and

obtaining a sleep staging result based on the box plot series.

Optionally, said obtaining EEG data to be processed comprises:

collecting a EEG signal in a single lead manner; and

performing down-sampling and filtering process on the EEG signal to obtain the EEG data to be processed.

Optionally, said obtaining a sleep staging result based on the box plot series comprises:

obtaining a numeric index range of the statistic characteristic values for each sleep staging; and

comparing the box plot series with the numeric index range to obtain the sleep staging result from the box plot series.

Optionally, the statistic characteristic values comprise at least a median and a inter quartile range (IQR).

Optionally, the sleep staging result comprises a deep sleep stage, a rapid eye movement (REM) stage, a light sleep stage and a wake stage;

in the deep sleep stage, the statistic characteristic values satisfy: 0.6≦median≦0.9 and IQR≦0.014;

in the REM stage, the statistic characteristic values satisfy: 0.375≦median≦0.45, or 0.275≦median≦0.375 and IQR≦0.014;

in the light sleep stage, the statistic characteristic values satisfy: 0.6≦median≦0.9 and IQR≦0.014, or 0.275≦median≦0.45 and IQR>0.014, or 0.45<median<0.6; and

in the wake stage, the statistic characteristic values satisfy: the median exceeds a first threshold and the IQR exceeds a second threshold, and both the median and the IQR are distributed randomly.

In one aspect, there is provided an EEG processing apparatus comprising:

a data obtaining unit configured to obtain EEG data to be processed;

a data analysis unit configured to analyze the EEG data to be processed to obtain a predetermined number of segments which do not overlap each other;

a data processing unit configured to obtain a maximum value and a minimum value of a peak-to-peak value in each of the segments, normalize a connection line between the maximum value and the minimum value of the peak-to-peak value in at least one of the segments to obtain upper end points and lower end points of the connection line, and calculate statistic characteristic values of an upper sideband curve formed by the upper end points to obtain a box plot series; and

a sleep staging unit configured to obtain a sleep staging result based on the box plot series from the data processing unit.

Optionally, the data obtaining unit comprises:

a signal collecting sub-unit configured to collect a EEG signal in a single lead manner; and

a signal pre-processing sub-unit configured to perform down-sampling and filtering process on the EEG signal to obtain the EEG data to be processed.

Optionally, the sleep staging unit comprises:

a numeric index obtaining sub-unit configured to obtain a numeric index range of the statistic characteristic values for each sleep staging; and

a sleep staging sub-unit configured to compare the box plot series with the numeric index range to obtain the sleep staging result from the box plot series.

In one aspect, there is provided a sleep monitoring wearable device comprise any EEG processing apparatus described in the above, wherein the EEG processing apparatus is configured to obtain a sleep staging result.

Optionally, the sleep monitoring wearable device further comprises a display apparatus for displaying the sleep staging result obtained by the EEG processing apparatus.

Optionally, the sleep monitoring wearable device further comprises a body temperature detecting apparatus configured to collect body temperature.

The display apparatus is also configured to display the body temperature collected by the temperature detecting apparatus.

Optionally, the sleep monitoring wearable device further comprises a heart rate collecting apparatus configured to collect heart rate.

The display apparatus is also configured to display the heart rate collected by the heart rate collecting apparatus.

Optionally, the sleep monitoring wearable device further comprises a blood oxygen collecting apparatus configured to collect blood oxygen saturation.

The display apparatus is also configured to display the blood oxygen saturation collected by the blood oxygen collecting apparatus.

Optionally, the sleep monitoring wearable device is a head band or a head cap.

In the above solutions, the following steps are performed: obtaining EEG data to be processed; analyzing the EEG data to be processed to obtain a predetermined number of segments which do not overlap each other; obtaining a maximum value and a minimum value of a peak-to-peak value in each of the segments; normalizing a connection line between the maximum value and the minimum value of the peak-to-peak value in each of the segments to obtain upper end points and lower end points of the connection line; calculating statistic characteristic values of an upper sideband curve formed by the upper end points in a framing manner to obtain a box plot series; and obtaining a sleep staging result based on the box plot series. In the solutions, the EEG data is subjected to a process on its amplitude to extract the amplitude information of the EEG data, and then the sleep is staged based on the amplitude information of the EEG data, which simplifies the sleep staging algorithm and reduces the complexity of the sleep staging.

### BRIEF DESCRIPTION OF THE DRAWINGS

In order to more clearly describe the technical solutions in the prior art or in embodiments of the present disclosure, the figures that may be used in the description on the embodiments or the prior art are briefly introduced in the following. Obviously, the figures in the following description are only some embodiments of the present disclosure. Those skilled in the art can obtain other figures based on these figures without creative work.

FIG. 1 is a schematic flowchart of an EEG processing method provided by an embodiment of the present disclosure;

FIG. 2 is a schematic flowchart of an EEG processing method provided by another embodiment of the present disclosure;

FIG. 3 is a schematic structural diagram of an EEG processing apparatus provided by an embodiment of the present disclosure;

FIG. 4 is a schematic structural diagram of an EEG processing apparatus provided by another embodiment of the present disclosure;

FIG. 5 is a schematic structural diagram of a sleep monitoring wearable device provided by an embodiment of the present disclosure; and

FIG. 6 is a schematic structural diagram of a sleep monitoring wearable device provided by another embodiment of the present disclosure.

### DETAILED DESCRIPTION

In the following, detailed description on EEG processing methods and apparatuses and sleep monitoring wearable devices provided by embodiments of the present disclosure will be made in connection with the attached figures, in which the same reference symbol indicate the same element in the specification. In the following description, in order to facilitate description, lots of details are provided to provide complete understanding of one or more embodiments. However, obviously, it is also possible implement the embodiments without these details.

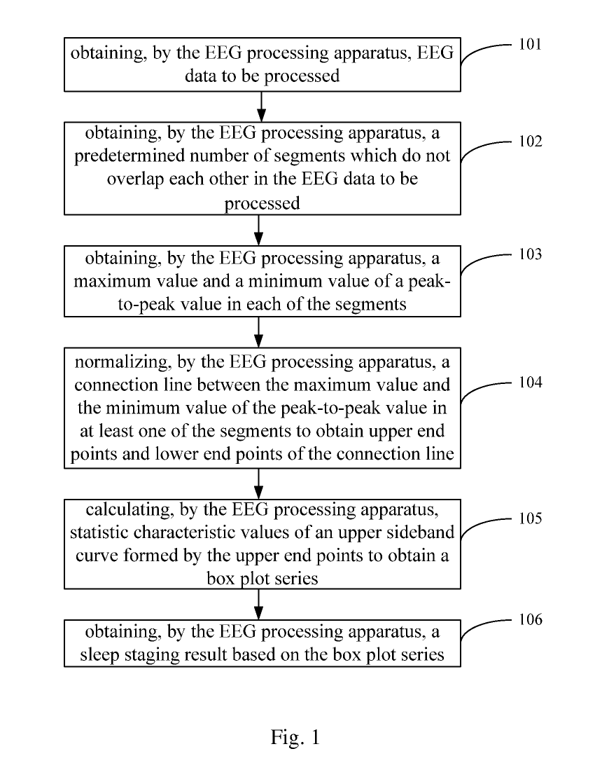

An embodiment of the present disclosure provides an EEG processing method, as illustrated in FIG. 1, which comprises the following steps:

At 101, obtaining EEG data to be processed by the EEG processing apparatus.

At 102, analyzing, by the EEG processing apparatus, the EEG data to be processed to obtain a predetermined number of segments which do not overlap each other.

In step 102, in the obtained predetermined number of segments, there is no limitation on the length of each segment. Specifically, in the step 102, the EEG data to be processed can be divided in time sequence, and the predetermined number of segments can be obtained in the segmented segments, wherein the obtained predetermined number of segments can be continuously or discontinuously distributed. In order to obtain an accurate sleep staging result, continuously distributed equal-length segments or uniformly distributed equal-length segments can be used here.

At 103, the EEG processing apparatus obtains a maximum value and a minimum value of a peak-to-peak value in each of the segments.

At 104, the EEG processing apparatus normalizes a connection line between the maximum value and the minimum value of the peak-to-peak value in at least one of the segments to obtain upper end points and lower end points of the connection line.

At 105, the EEG processing apparatus calculates statistic characteristic values of an upper sideband curve formed by the upper end points to obtain a box plot series.

In the step 105, for the statistic characteristic values of the upper sideband curve formed by the upper end points, a framing calculation manner can be employed to obtain the box plot series. Since the time length of each frame is equal in the framing calculation manner, the time axis of the obtained box plot series represents reference quantities with equal distance, easing subsequent processing in the following steps.

The statistic characteristic values comprise at least a median and a inter quartile range (IQR).

At 106, the EEG processing apparatus obtains a sleep staging result based on the box plot series.

In the above solution, the following steps are performed: obtaining EEG data to be processed; analyzing the EEG data to be processed to obtain a predetermined number of segments which do not overlap each other; obtaining a maximum value and a minimum value of a peak-to-peak value in each of the segments; normalizing a connection line between the maximum value and the minimum value of the peak-to-peak value in each of the segments to obtain upper end points and lower end points of the connection line; calculating statistic characteristic values of an upper sideband curve formed by the upper end points in a framing manner to obtain a box plot series; and obtaining a sleep staging result based on the box plot series. In the solutions, the EEG data is subjected to a process on its amplitude to extract the amplitude information of the EEG data, and then the sleep is staged based on the amplitude information of the EEG data, which simplifies the sleep staging algorithm and reduces the complexity of the sleep staging.

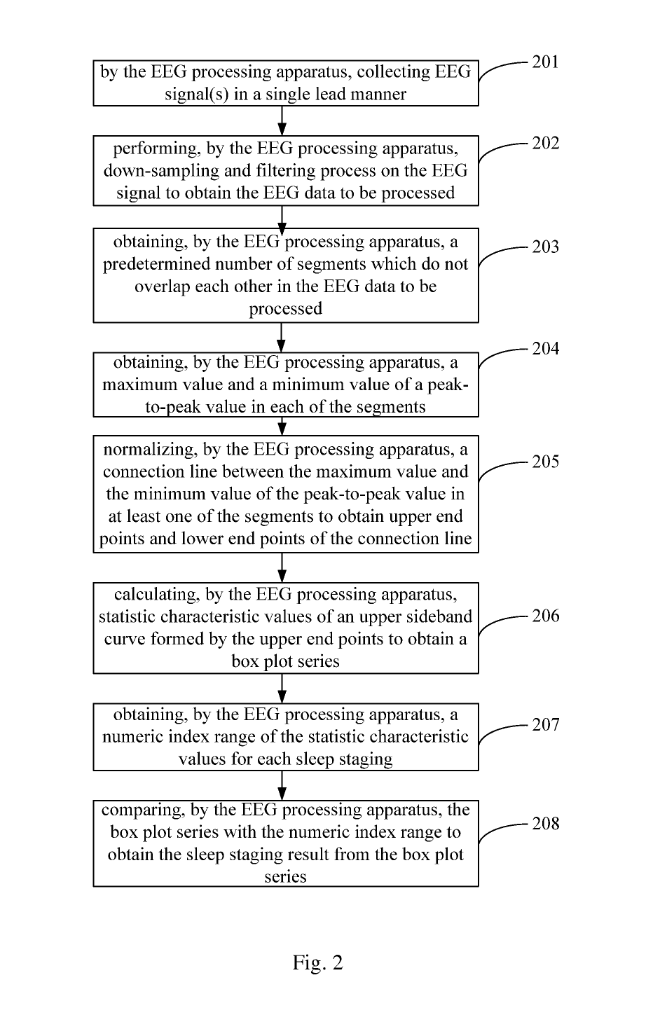

In particular, as illustrated in FIG. 2, an embodiment of the present disclosure provides an EEG processing method, wherein,

At 201, the EEG processing apparatus collects EEG signal(s) in a single lead manner.

In particular, collecting EEG signal(s) in a single lead manner can comprise: performing collection by three electrodes arranged at the prefrontal near the positions of Fp1-Fp2, where Fp1 is the left prefrontal sampling point and Fp2 is the right prefrontal sampling point. In step 201, sampling can be performed in a high frequency to reflect real EEG signal in the sleep as much as possible.

At 202, the EEG processing apparatus performs down-sampling and filtering process on the EEG signal to obtain the EEG data to be processed.

In step 202, in order to improve the processing speed, it is possible to reduce the data density to be processed through down-sampling. For example, when the sampling frequency in step 201 is 1000 Hz, it becomes 100 Hz through the down-sampling process of the algorithm in step 202. In step 202, the filtering process eliminates the baseline drift and eliminates the influence due to sweating, electro disturbance, muscle motion, turn-over of the body or the like, while preserving the originally collected EEG signals to the largest extent.

At 203, the EEG processing apparatus analyzes the EEG data to be processed to obtain a predetermined number of segments which do not overlap each other.

In step 203, in the obtained predetermined number of segments, there is no limitation on the length of each segment herein. Specifically, in the step 203, the EEG data to be processed can be divided in time sequence, and the predetermined number of segments can be obtained in the segmented segments, wherein the obtained predetermined number of segments can be continuously or discontinuously distributed. In order to obtain an accurate sleep staging result, continuously distributed equal-length segments or uniformly distributed equal-length segments can be used here. In the step 203, there is no limitation on the length of the segmented predetermined number of segments. One way is to use 20 seconds per segment.

At 204, the EEG processing apparatus obtains a maximum value and a minimum value of a peak-to-peak value in each of the segments.

At 205, the EEG processing apparatus normalizes a connection line between the maximum value and the minimum value of the peak-to-peak value in at least one of the segments to obtain upper end points and lower end points of the connection line.

The normalizing a connection line between the maximum value and the minimum value of the peak-to-peak value in at least one of the segments in step 205 is: performing normalizing process in the vertical axis of the coordinate system to eliminate the difference between different EEG signals in the at least one segment by the normalizing process to facilitate the subsequent uniform calculation. Of course, for the segments that are not normalized, no subsequent process is needed; therefore, the result is not subjected to substantial influence. Naturally, an optional manner is to normalize the connection line between the maximum value and the minimum value of the peak-to-peak value in all the predetermined number of the segments in step 203, in order to improve the precision of the sleep staging result.

At 206, the EEG processing apparatus calculates statistic characteristic values of an upper sideband curve formed by the upper end points to obtain a box plot series.

In the step 206, for the statistic characteristic values of the upper sideband curve formed by the upper end points, a framing calculation manner is employed to obtain the box plot series. Since the time length of each frame is equal in the framing calculation manner, the time axis of the obtained box plot series represents reference quantities with equal distance, easing subsequent processing in the following steps.

At 207, the EEG processing apparatus obtains a numeric index range of the statistic characteristic values for each sleep staging.

The numeric index range of the statistic characteristic values can be a preset numeric index range in the apparatus. The numeric range is obtained in advance by experiments based on multiple groups of people. The statistic characteristic values comprise at least a median and a inter quartile range (IQR).

At 208, the EEG processing apparatus compares the box plot series with the numeric index range to obtain the sleep staging result from the box plot series.

In step 208, the sleep staging result comprises a deep sleep stage, a rapid eye movement (REM) stage, a light sleep stage and a wake stage. The REM is also called Para-sleep or fast wave sleep. In the deep sleep stage, the statistic characteristic values satisfy: 0.6≦median≦0.9 and IQR≦0.014; in the REM stage, the statistic characteristic values satisfy: 0.375≦median≦0.45, or 0.275≦median≦0.375 and IQR≦0.014; in the light sleep stage, the statistic characteristic values satisfy: 0.6≦median≦0.9 and IQR>0.014, or 0.275≦median≦0.45 and IQR>0.014, or 0.45<median<0.6; and in the wake stage, the statistic characteristic values satisfy the following: the median exceeds a first threshold and the IQR exceeds a second threshold, and both the median and the IQR are distributed randomly.

In the above solution, the following steps are performed: obtaining EEG data to be processed; analyzing the EEG data to be processed to obtain a predetermined number of segments which do not overlap each other; obtaining a maximum value and a minimum value of a peak-to-peak value in each of the segments; normalizing a connection line between the maximum value and the minimum value of the peak-to-peak value in each of the segments to obtain upper end points and lower end points of the connection line; calculating statistic characteristic values of an upper sideband curve formed by the upper end points in a framing manner to obtain a box plot series; and obtaining a sleep staging result based on the box plot series. In the solutions, the EEG data is subjected to a process on its amplitude to extract the amplitude information of the EEG data, and then the sleep is staged based on the amplitude information of the EEG data, which simplifies the sleep staging algorithm and reduces the complexity of the sleep staging.

As illustrated in FIG. 3, an embodiment of the present disclosure provides an EEG processing apparatus comprising:

a data obtaining unit 31 configured to obtain EEG data to be processed;

a data analysis unit 32 configured to analyze the EEG data to be processed to obtain a predetermined number of segments which do not overlap each other;

a data processing unit 33 configured to obtain a maximum value and a minimum value of a peak-to-peak value in each of the segments, normalize a connection line between the maximum value and the minimum value of the peak-to-peak value in at least one of the segments to obtain upper end points and lower end points of the connection line, and calculate statistic characteristic values of an upper sideband curve formed by the upper end points to obtain a box plot series; and

a sleep staging unit 34 configured to obtain a sleep staging result based on the box plot series.

In the above solution, the EEG processing apparatus obtains EEG data to be processed; analyzes the EEG data to be processed to obtain a predetermined number of segments which do not overlap each other; obtains a maximum value and a minimum value of a peak-to-peak value in each of the segments; normalizes a connection line between the maximum value and the minimum value of the peak-to-peak value in each of the segments to obtain upper end points and lower end points of the connection line; calculates statistic characteristic values of an upper sideband curve formed by the upper end points in a framing manner to obtain a box plot series; and obtaining a sleep staging result based on the box plot series. In the solutions, the EEG data is subjected to a process on its amplitude to extract the amplitude information of the EEG data, and then the sleep is staged based on the amplitude information of the EEG data, which simplifies the sleep staging algorithm and reduces the complexity of the sleep staging.

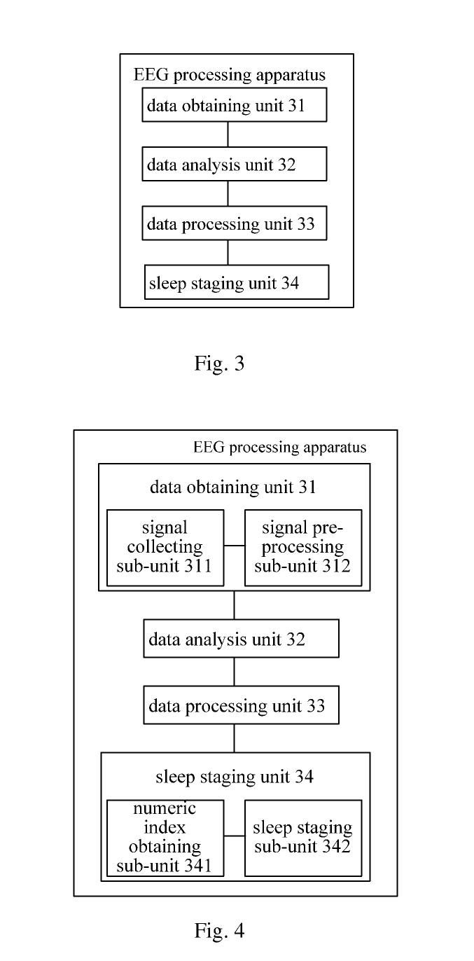

As illustrated in FIG. 4, optionally, the data obtaining unit 31 comprises:

a signal collecting sub-unit 311 configured to collect a EEG signal in a single lead manner; and

a signal pre-processing sub-unit 312 configured to perform down-sampling and filtering process on the EEG signal to obtain the EEG data to be processed.

In order to improve the processing speed, it is possible to reduce the data density to be processed through down-sampling. For example, when the sampling frequency of the signal sampling sub-unit 311 is 1000 Hz, the signal pre-processing sub-unit 312 changes the sample frequency to 100 Hz through the down-sampling process of an algorithm. By the filtering process, the signal pre-processing sub-unit 312 eliminates the baseline drift and eliminates the influence due to sweating, electro disturbance, muscle motion, turning-over of body or the like, while preserving the originally collected EEG signals to the largest extent.

As illustrated in FIG. 4, optionally, the sleep staging unit 34 comprises:

a numeric index obtaining sub-unit 341 configured to obtain a numeric index range of the statistic characteristic values for each sleep staging; and

a sleep staging sub-unit 342 configured to compare the box plot series with the numeric index range obtained by the numeric index obtaining sub-unit 341 to obtain the sleep staging result from the box plot series.

The numeric index range of the statistic characteristic values can be a preset numeric index range in the apparatus. The numeric range can be obtained in advance by experiments based on multiple groups of people. The statistic characteristic values comprise at least a median and a inter quartile range (IQR). The sleep staging result comprises a deep sleep stage, a rapid eye movement (REM) stage, a light sleep stage and a wake stage. In the deep sleep stage, the statistic characteristic values satisfy: 0.6≦median≦0.9 and IQR≦0.014; in the REM stage, the statistic characteristic values satisfy: 0.375≦median≦0.45, or 0.275≦median≦0.375 and IQR≦0.014; in the light sleep stage, the statistic characteristic values satisfy: 0.6≦median≦0.9 and IQR>0.014, or 0.275≦median≦0.45 and IQR>0.014, or 0.45<median<0.6; and in the wake stage, the statistic characteristic values satisfy: the median exceeds a first threshold and the IQR exceeds a second threshold, where both the first threshold and the second threshold are large, for example, the first threshold can be 0.9 and the second threshold can be 0.014, and both the median and the IQR are distributed randomly.

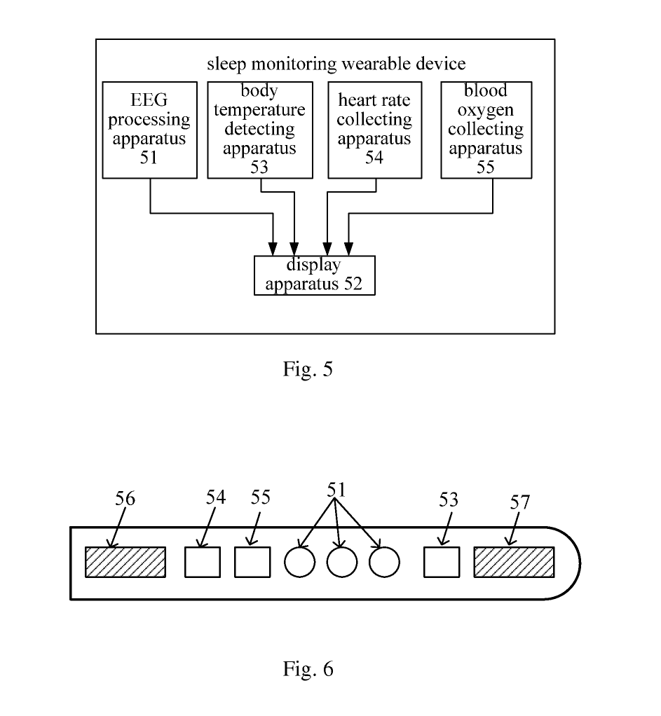

As illustrated in FIG. 5, an embodiment of the present disclosure also provides a sleep monitoring wearable device comprise any EEG processing apparatus 51 described in the above, wherein the EEG processing apparatus is configured to obtain a sleep staging result.

Optionally, as illustrated in FIG. 5, the sleep monitoring wearable device further comprises a display apparatus 52 for displaying the sleep staging result obtained by the EEG processing apparatus.

Optionally, as illustrated in FIG. 5, the sleep monitoring wearable device further comprises a body temperature detecting apparatus 53 configured to collect body temperature.

The display apparatus 52 is also configured to display the body temperature collected by the temperature detecting apparatus 53.

Optionally, as illustrated in FIG. 5, the sleep monitoring wearable device further comprises a heart rate collecting apparatus 54 configured to collect heart rate.

The display apparatus 52 is also configured to display the heart rate collected by the heart rate collecting apparatus 54.

Optionally, as illustrated in FIG. 5, the sleep monitoring wearable device further comprises a blood oxygen collecting apparatus 55 configured to collect blood oxygen saturation.

The display apparatus 52 is also configured to display blood oxygen saturation collected by the blood oxygen collecting apparatus 55.

The temperature has a large effect on the sleep quality, such as the rising of temperature can accelerate sleep, but the decreasing of skin temperature after being asleep helps remain good sleep. Monitoring sleep temperature largely helps monitoring diabetes. In addition, monitoring the heart rate and the blood oxygen saturation (SpO2) can effectively monitor related diseases. The sleep monitoring wearable device provided by an embodiment of the present disclosure can monitor the temperature, the heart rate and the blood oxygen saturation simultaneously in real time. In particular, the body temperature detecting apparatus can use a flexible thermosensitive resistor for collecting temperature. The heart rate collecting apparatus and the blood oxygen collecting apparatus can use a conventional heart rate/SpO2 optoelectronic measurement module for collecting and processing the data.

Optionally, the sleep monitoring wearable device is a headband or a head cap. As illustrated in FIG. 6, description is made by taking the headband as example. The head band can be a closed circular ring, or a band as illustrated in FIG. 6. Of course, the wearing manner of the head band can be tied or buckled, such as being attached by a hook and loop fastener, a snap fastener, a button fastener, a flexible attachable material or the like. The band-shape sleep monitoring wearable device illustrated in FIG. 6 comprises an EEG processing apparatus 51, a body temperature detecting apparatus 53, a heart rate collecting apparatus 54, and a blood oxygen collecting apparatus 55. The figure also illustrates flexible attachable materials 56 and 57 capable of being attached for wearing of the device. The EEG processing apparatus 51, the body temperature detecting apparatus 53, the heart rate collecting apparatus 54 and the blood oxygen collecting apparatus 55 contact the human body directly. The specific positional relation of the EEG processing apparatus 51, the body temperature detecting apparatus 53, the heart rate collecting apparatus 54 and the blood oxygen collecting apparatus 55 is not limited in the present disclosure, as long as the EEG processing apparatus 51 can be fixed to the prefrontal near the positions of Fp1-Fp2 through the sampling electrodes when being worn. FIG. 6 schematically illustrates the EEG processing apparatus 51 by three circle electrodes. The display apparatus 53 does not contacts the human body directly due to its display function; therefore, it can be arranged on the other side of the head band, which is not illustrated in the figure.

In connection with the above manner, since the sleep monitoring wearable device is a head band or a head cap, it is possible to directly adopt the wearing manner to fix the sampling electrodes using the single lead manner to collect EEG signals to the prefrontal near the positions of Fp1-Fp2, and it is possible to use a disposable electrode, to avoid influence on sleep quality due to the fact that it needs to daub conductive adhesive for placing the sampling electrodes. In addition, the wearable sleep monitoring device is easy to be put on, and it can avoid falling off of the electrodes during sleep.

The above descriptions are only specific implementations of the present disclosure, but the protection scope of the present disclosure is not limited to this. Changes or replacements that can be easily devised by those skilled in the art within the technical scope of the present disclosure should all fall within the protection scope of the present disclosure. Therefore, the protection scope of the present disclosure should be defined by the protection scope of the claims.

The present application claims the priority of Chinese Patent Application No. 201410490680.X filed on Sep. 23, 2014, entire content of which is incorporated as a part of the present application by reference.

1. A method for processing an electroencephalogram EEG, comprising:
obtaining EEG data to be processed;
analyzing the EEG data to be processed to obtain a predetermined number of segments which do not overlap each other;
obtaining a maximum value and a minimum value of a peak-to-peak value in each of the segments;
normalizing a connection line between the maximum value and the minimum value of the peak-to-peak value in at least one of the segments to obtain upper end points and lower end points of the connection line;
calculating statistic characteristic values of an upper sideband curve formed by the upper end points to obtain a box plot series; and
obtaining a sleep staging result based on the box plot series.

2. The method according to claim 1, wherein said obtaining EEG data to be processed comprises:
collecting an EEG signal in a single lead manner; and
performing a down-sampling and filtering process on the EEG signal to obtain the EEG data to be processed.

3. The method according to claim 1, wherein said obtaining the sleep staging result based on the box plot series comprises:
obtaining a numeric index range of the statistic characteristic values for each sleep staging; and
comparing the box plot series with the numeric index range to obtain the sleep staging result from the box plot series.

4. The method according to claim 1, wherein the statistic characteristic values comprise at least a median and an inter quartile range IQR.

5. The method according to claim 4, wherein the sleep staging result comprises a deep sleep stage, a rapid eye movement (REM) stage, a light sleep stage and a wake stage;
in the deep sleep stage, the statistic characteristic values satisfy: 0.6≦median≦0.9 and IQR≦0.014;
in the REM stage, the statistic characteristic values satisfy: 0.375≦median≦0.45, or 0.275≦median≦0.375 and IQR≦0.014;
in the light sleep stage, the statistic characteristic values satisfy one of the following:
0.6≦median≦0.9 and IQR>0.014; 0.275≦median≦0.45 and IQR>0.014; and 0.45<median<0.6; and
in the wake stage, the statistic characteristic values satisfy: the median exceeds a first threshold and the IQR exceeds a second threshold, and both the median and the IQR are distributed randomly.

6. The method according to claim 1, wherein said analyzing the EEG data to be processed to obtain the predetermined number of segments comprises:
dividing the EEG data to be processed in time sequence, and obtaining the predetermined number of segments in the divided segments, wherein the obtained predetermined number of segments are continuously or discontinuously distributed.

7. An apparatus for processing an electroencephalogram EEG, comprising:
a data obtaining unit configured to obtain EEG data to be processed;
a data analysis unit configured to analyze the EEG data to be processed to obtain a predetermined number of segments which do not overlap each other;
a data processing unit configured to obtain a maximum value and a minimum value of a peak-to-peak value in each of the segments, normalize a connection line between the maximum value and the minimum value of the peak-to-peak value in at least one of the segments to obtain upper end points and lower end points of the connection line, and calculate statistic characteristic values of an upper sideband curve formed by the upper end points to obtain a box plot series; and
a sleep staging unit configured to obtain a sleep staging result based on the box plot series.

8. The apparatus according to claim 7, wherein the data obtaining unit comprises:
a signal collecting sub-unit configured to collect a EEG signal in a single lead manner; and
a signal pre-processing sub-unit configured to perform a down-sampling and filtering process on the EEG signal to obtain the EEG data to be processed.

9. The apparatus according to claim 7, wherein the sleep staging unit comprises:
a numeric index obtaining sub-unit configured to obtain a numeric index range of the statistic characteristic values for each sleep staging; and
a sleep staging sub-unit configured to compare the box plot series with the numeric index range to obtain the sleep staging result from the box plot series.

10. A sleep monitoring wearable device comprises the apparatus for processing EEG according to claim 7, wherein the apparatus for processing EEG is configured to obtain the sleep staging result.

11. The sleep monitoring wearable device according to claim 10, further comprising a body temperature detecting apparatus configured to collect body temperature.

12. The sleep monitoring wearable device according to claim 10, further comprising a heart rate collecting apparatus configured to collect heart rate.

13. The sleep monitoring wearable device according to claim 10, further comprising a blood oxygen collecting apparatus configured to collect blood oxygen saturation.

14. The sleep monitoring wearable device according to claim 10, further comprising a display apparatus, wherein the display apparatus is configured to display at least one of the sleep staging result, the body temperature, the heart rate and the blood oxygen saturation.

15. The sleep monitoring wearable device according to claim 10, wherein the sleep monitoring wearable device is a head band or a head cap.

16. The sleep monitoring wearable device according to claim 10, wherein the data obtaining unit comprises:
a signal collecting sub-unit configured to collect a EEG signal in a single lead manner; and
a signal pre-processing sub-unit configured to perform a down-sampling and filtering process on the EEG signal to obtain the EEG data to be processed.

17. The sleep monitoring wearable device according to claim 10, wherein the sleep staging unit comprises:
a numeric index obtaining sub-unit configured to obtain a numeric index range of the statistic characteristic values for each sleep staging; and
a sleep staging sub-unit configured to compare the box plot series with the numeric index range to obtain the sleep staging result from the box plot series.

18. (canceled)

19. (canceled)
